(19) United States
(12) Patent Application Publication
(10) Pub. No.: US 2026/0256498 A1
(43) Pub. Date: Sep. 3, 2026

(54) BI-LATERAL PELVIC STABILIZATION

(71) Applicant: SI-Bone Inc., Santa Clara, CA (US)

(72) Inventors: Bradley J. ANDERSON, Jr., Alexandria, MN (US); W. Carlton RECKLING, Cheyenne, WY (US); Scott A. YERBY, Montara, CA (US); Paul M. SAND, Redwood City, CA (US); Bret W. SCHNEIDER, San Jose, CA (US); Francois FOLLINI, Austin, TX (US)

(21) Appl. No.: 18/870,896

(22) PCT Filed: Jun. 1, 2023
(86) PCT No.: PCT/US2023/067747

§ 371 Date: Dec. 2, 2024

Related U.S. Application Data
(60) Provisional application No. 63/365,674, filed on Jun. 1, 2022.

(51) Int. Cl. A61B 17/68; A61B 17/86

(52) U.S. Cl. CPC A61B 17/683; A61B 17/8635; A61B 17/864; A61B 17/8665; A61B 2017/681

(57) ABSTRACT

Methods of stabilizing a pelvis, optionally a pelvic fracture, which may also be referred to herein as bilateral pelvic stabilization. Optionally, the stabilization may include applying compression across the pelvis between and across the sacro-iliac (SI) joints. Stabilization (and optionally compression) implants herein are generally sized and configured to extend across both SI joints and the sacrum, and extend at least partially within the ilia.

### CROSS REFERENCE TO RELATED APPLICATIONS AND INCORPORATION BY REFERENCE

This application claims priority to U.S. Prov. App. No. 63/365,674, filed Jun. 1, 2022, the entire disclosure of which is incorporated by reference herein in its entirety for all purposes.

U.S. Pat. No. 10,363,140, is incorporated by reference herein in its entirety for all purposes, including FIGS. 88A-90B and the descriptions thereof. This application also incorporates by reference herein for all purposes the disclosures of the following: U.S. Pat. Nos. 9,011,501; 9,408,637; and 6,635,059. Any relevant disclosure from these references that are incorporated by reference herein may be included in this disclosure. For example, without limitation, disclosure in U.S. Pat. No. 10,363,140 related to preparing a hole or opening in tissue for an implant may be incorporated by reference herein in methods of implantation.

### BACKGROUND

There may be benefits to stabilizing a pelvic fracture, which can occur due to injury. There may be benefits to stabilizing a pelvic fracture along a common axis and/or a with single implant. Additionally, there may be benefits to applying compression along a common axis to stabilize a pelvic fracture.

### SUMMARY OF THE DISCLOSURE

One aspect of the disclosure is a method of one or both of pelvic stabilization or bilateral sacro-iliac joint stabilization.

In this aspect, the method optionally includes laterally implanting an implant in a first ilium, across a first SI joint, through an S1, S2 or S3 sacrum corridor, across a contralateral second SI joint, and in a second ilium.

In this aspect, the method optionally includes applying compression across the first and second SI joints with the implant.

In this aspect, implanting the implant optionally comprises advancing a first component of the implant from a first lateral side and advancing a second component of the implant from a second lateral side, optionally coupling the first component to the second component. Implanting the implant optionally further comprises engaging an outer external thread on the first component with an internal thread on the second component. A second component optionally extends across a midline of the sacrum. A first component optionally extends across the midline of the sacrum. The implanting step optionally implants an entirety of a first component on a first side of a midline of the sacrum. The implanting step optionally implants an entirety of a second component on a second side of a midline of the sacrum that is contralateral to a first side of the midline.

In this aspect, the implanting step optionally further comprises implanting a third component laterally from a first lateral side, through a first component, out of a distal end of the first component, and into engagement with a second component. The method optionally further comprising engaging an outer thread of a third component with a second component inner thread. A first component and a second component optionally have greater radially outermost dimensions from respective long axes than an outermost radial dimension of a third component.

In this aspect, a first component of the implant optionally has a greater radially outermost dimension than a radially outermost dimension of a second component of the implant.

One aspect of the disclosure is an implant sized and configured to be implanted across both sacro-iliac joints in a patient, which may be referred to as a pelvic implant.

In this aspect, the implant optionally includes a plurality of components.

In this aspect, the implant optionally include stabilization means for bilateral stabilization that extends across first and second of a patient's sacro-iliac joints and through one of an S1 corridor, an S2 corridor, or an S3 corridor.

In this aspect, a stabilization means optionally has a length from 125 mm to 145 mm.

In this aspect, a stabilization means optionally comprises a first component and a second component, the first component having a distal end that is sized and configured to be advanced into a distal end of the second component and coupled thereto. A first component optionally includes an outer thread, and a second component optionally comprises an internal thread.

In this aspect, a stabilization means optionally comprises a first component, a second component, and a third component, wherein the third component is optionally sized and configured to extend through the first and second components, and wherein a distal region of the third component is optionally sized and configured to be coupled to an inner region of the second component.

In this aspect, a stabilization means may further comprise compression means for applying compression across and between the first and second sacro-iliac joints.

In this aspect, the implant optionally includes compression means for applying bilateral compression across first and second sacro-iliac joints and through one of an S1 corridor, an S2 corridor, or an S3 corridor that is between the sacro-iliac joints.

### BRIEF DESCRIPTION OF THE DRAWINGS

FIG. 1A is an anterior view illustrating an exemplary implantation axis passing through an S2 corridor.

FIG. 1B is a side sectional view illustrating an exemplary implantation axis passing through an S2 corridor.

FIG. 2A is an anterior view illustrating an exemplary implantation axis passing through an S1 corridor.

FIG. 2B is a side sectional view illustrating an exemplary implantation axis passing through an S1 corridor.

FIG. 3 is a posterior view of a portion of a pelvis representing an implant location that is implanted across both SI joints and extending through an S2 corridor.

FIG. 4 is a representative view of a representation of a sacrum and an exemplary implant (comprising a plurality of components) implanted across the sacrum through a sacrum corridor.

FIG. 5A is a representative view of a representation of a sacrum and an exemplary implant (comprising first, second and third components) implanted across the sacrum through a sacrum corridor.

FIG. 5B illustrates the implant of FIG. 5A, showing in dashed lines internal surfaces of the components.

FIG. 5C represents the internal component in FIGS. 5A and 5B.

FIG. 6 illustrates an alternative general approach in which a threaded anchor (shown at the left in the figure) that is coupled to a cable extending proximally from the anchor (or other similar elongate member) is delivered through a first ilium, across an SI joint, through the sacrum, across the contralateral SI joint, and into the contralateral ilium, where the anchor is fastened and implanted.

FIG. 7 illustrates an alternative implant system that is adapted for bi-lateral SI-joint placement and can be used to stabilize the pelvis, and which may be implanted in any of the locations described herein (e.g., along an axis that extends through an S1 corridor, an S2 corridor, or an S3 corridor).

### DETAILED DESCRIPTION

There may be benefits to stabilizing a pelvic fracture, which can occur due to injury. There may be benefits to stabilizing a pelvic fracture along a common axis and/or a with single implant. Additionally, there may be benefits to applying compression along a common axis to stabilize a pelvic fracture. For example, a common axis may extend through a first ilium, through a first SI joint, through the sacrum, through the second SI joint, and through the second ilium. As used in this context, a single implant can include one or more components that when assembled or coupled together create or define the implant. “Coupled” as used in this regard does not necessary impart any particular mechanical coupling, but may include multiple components that are directly or indirectly secured together. The components together may also be referred to herein as a system, an assembly, or other similar term. It is of course understood that multiple implants may be implanted in different corridor locations as part of an overall therapy (each of which may comprise one or more individual components). The methods of, and devices adapted for, stabilizing pelvic fractures herein can provide the additional benefit of fusing both sacroiliac (“SI”) joints. In some applications, the implants may be used primarily to fuse the SI joints.

Methods of stabilizing a pelvic fracture as described herein may be referred to herein as bilateral stabilization, and optionally bilaterally stabilization with compression if compressive forces are applied across the SI joints. Implants herein are generally sized and configured to extend across both SI joints and the sacrum, and extend at least partially within the ilia.

While the disclosure herein generally focuses on methods of, and implants adapted for, applying compression across both SI joints, there may be applications in which compression may not be necessary or essential, and the disclosure herein related to bilateral stabilization with a single implant and/or along a common axis may still apply in these applications.

The bilateral stabilization implants herein may be adapted to be delivered along a trajectory and implanted along an axis that is within one of an S1 level corridor, an S2 level corridor, or an S3 level corridor. Optionally, each of a plurality of implants may be delivered and implanted along an axis that is within one of an S1 level corridor, an S2 level corridor, or an S3 level corridor. The delivery trajectories and axes of implantation also extend through both SI joints and at least partially into the ilia.

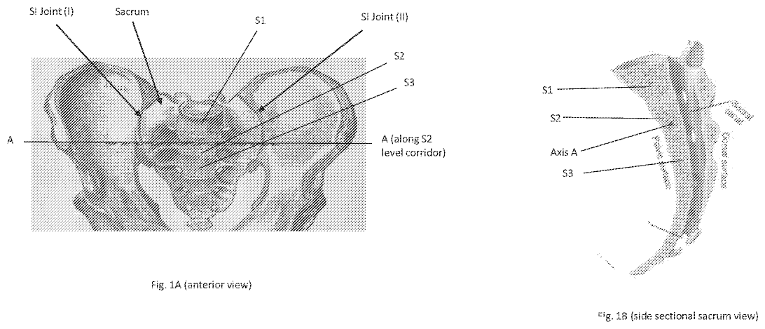

FIGS. 1A and 1B illustrate an exemplary implantation axis A-A, a part of which is within the S2 level corridor of the sacrum, as shown. Axis A-A is also the general delivery trajectory for one or more components of the implant. For some multi-part implants herein, one part may be delivered along a first trajectory from one lateral side (e.g., left to right in FIG. 1A) while another part may be delivered along a second trajectory from a second lateral side (e.g., right to left in FIG. 1A), wherein both trajectories are aligned (or at least generally aligned) and generally include a common axis (e.g., axis A-A), which may also be the axis of implantation.

FIG. 1B illustrates a lateral sectional view of the sacrum, labeling the sacral canal and the S1-S3 vertebrae of the sacrum. Axis A-A shown in FIG. 1A is labeled in FIG. 1B, illustrating the axis A-A extending or passing through the S2 level corridor. As can be seen and appreciated, the delivery trajectories and axis of implantation need to avoid the sacral canal and the posterior and anterior foramen to avoid damaging nerves and other sensitive tissue and vessels (e.g., arteries, veins, etc.).

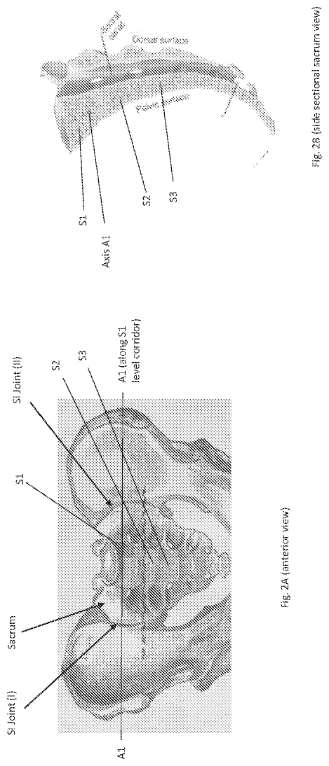

FIGS. 2A and 2B illustrate an exemplary axis/trajectory A1-A1 extending or passing through an S1 level corridor, as shown. Similarly, while not labeled, a trajectory and/or axis may extend or pass through an S3 level corridor. In some embodiments, for example only, a first implant may be delivered and implanted along axis A1-A1 in the S1 level corridor and a second implant may be delivered and implanted along axis A-A in the S2 level corridor.

In general, the boundaries of the S1, S2 and S3 level corridors through which an implant may safely and effectively be delivered and implanted are small, or narrow, meaning there is not a large volume of bone in the corridor that is available to safely receive the implant. The implant that passes within and through the boundaries of the corridor must therefore be small enough in overall profile so as to stay within the boundaries and not damage sensitive tissue, yet the overall implant must also be strong enough to withstand forces thereon once implanted, which may be relatively larger along the midline of the sacrum. Additionally, in general, the regions of the implant that are generally laterally outside of the corridor(s) are preferably sized and configured to help anchor the implant in bone and facilitate at least one of ingrowth, on growth or throughgrowth, which helps stabilize the implant after implantation. The implants herein are therefore preferably sized and configured such that 1) a portion of the implant safely extends along one of the corridors across a midline of the spine; 2) the implant is strong enough to withstand the forces acting thereon, which may be concentrated or greatest at one or more locations; 3) at least a portion of the implant facilitates tissue growth (e.g., bone) to enhance stabilization of the implant once implanted; and 4) is properly and safely anchored in place in one or more locations along its length. Not every one of these considerations may, however, be essential, depending on the particular configuration of the overall implant and/or the therapy that is needed, and thus implants herein may conceivable be adapted to provide less than all of these functions.

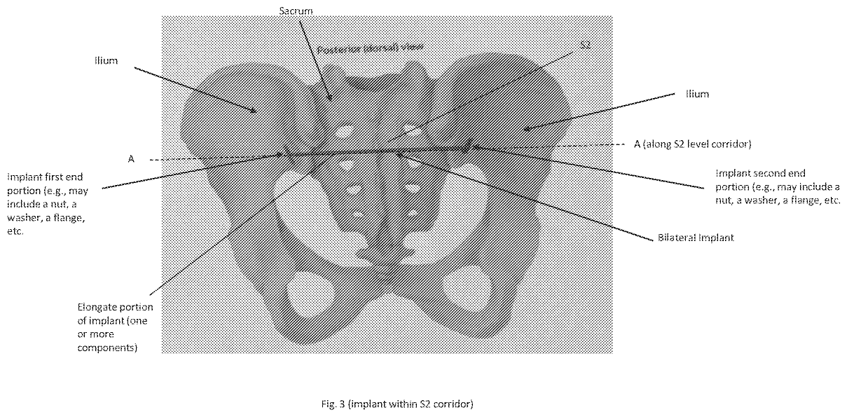

FIG. 3 is a posterior view illustrating a position of a merely exemplary bilateral implant after implantation, and may be illustrative of an exemplary implantation axis/trajectory A-A, a portion of which extends within the boundaries of the S2 corridor in this example. As shown, the axis also passes through both SI joints, and within both ilia. The main elongate body portion is shown in FIG. 3 to illustrate its position relative to the surrounding anatomy, but it is understood that while the first and second end portions extend out of the ilia (proud of the ilia) to some extent, the elongate main body portion is at least mostly disposed within the ilia, across the SI joints, and within the corridor of the sacrum at the level of implantation (S2 in this example).

While the corridor through which the implant is positioned may be relatively small, the anatomical regions that are lateral to the foramen and that lie along the axis of implantation are generally able to accept larger portions of the implant. There may thus be more design freedom for the regions of the implants that are intended to be disposed in anatomical regions that are lateral to the foramen, which includes portions of the sacrum, the SI joints, and portions of the ilia. This may allow the lateral implant portions to be designed with functionality that may not be possible or as easily designable for the portion of the implant at the lateral position of and between the foramen. A “central” portion of the implants herein may refer to a portion of the implant that is intended to be implanted laterally between the foramen within a corridor and that extends across the sacral midline. “Lateral” portions of the implants herein may refer to portions of the implant that are, or at least a portion of which are, intended to be implanted laterally relative to foramen in its axis of implantation, such as at least partially in the sacral alae. The central and lateral portions of the implants herein may have different configurations and/or may provide different functionality for the implant. For example only and without any limitations, a central portion of the implant may beneficially have a relatively smaller outer dimension or profile compared with lateral implant portions (so that it can be safely implanted within the corridor). Additionally, and for example only and without any limitations, lateral portions or at least a portion of the lateral portions of the implant may be relatively larger than the central portion, may provide relatively more anchoring or stabilization functionality for the implant, and may optionally include one or more features that facilitates at least one of ingrowth, on-growth, or through-growth, such as in regions that are disposed across the SI joints.

It may be challenging to implement an effective relatively small-profile and one-piece elongate body (that includes the central portion of the implant) that provides all of the desired or necessary functionality of a bilateral SI joint stabilization implant, at least along the length of the elongate body portion of the implant. Some of the implants herein may include multiple components that when assembled create or define the overall implant. An exemplary but optional advantage of a multi-piece bilateral SI joint stabilization implant is that a first part of the implant may be sized and configured to provide one or more functions while a second part of the implant may be sized and configured to provide one or more functions, any of which may be different that the functions provided by the first part. When assembled, however, (which may occur during the procedure or before), the assembled implant has the desired overall functionality. For example only, and without limitation, an inner elongate member may have a smaller outer diameter or profile and may be made of relatively stronger material than one or more outer elongate members through which the inner elongate member extends, and one or more outer elongate members may be adapted to provide better anchoring and/or promote better tissue growth than an inner member. In some non-limiting examples, a relatively strong but small-profile inner member may provide more of the needed strength, but its small profile or size may allow for larger lateral portions that are better configured for stabilization and/or tissue growth to be safely and effectively implanted in the lateral regions.

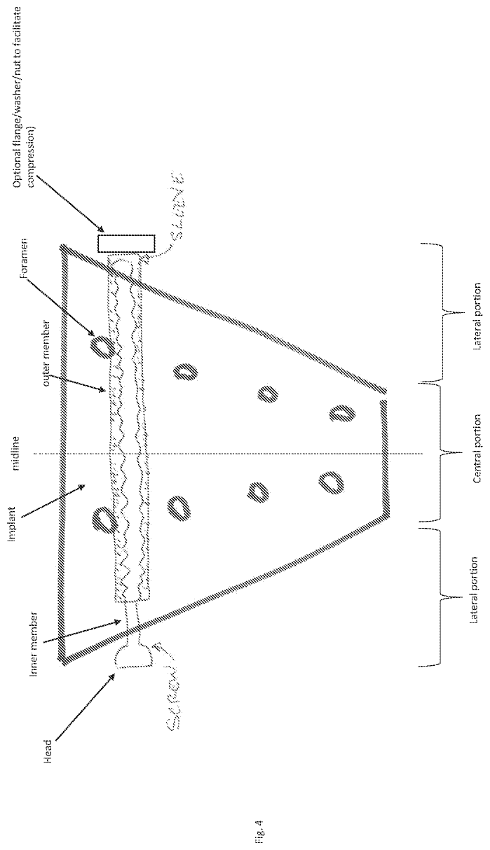

FIG. 4 illustrates a portion of a merely exemplary implant and an exemplary implantation location along an S2 corridor, and a figure illustrative of a sacrum (the SI joints and ilia are not shown for clarity). It is understood that the elongate body portion of the implant within the sacrum would not be visible after implantation. Exemplary and general implant central portion and implant lateral portions are also labeled, the description of which may be applicable to all implants herein. In this exemplary embodiment the inner member includes a head at one end (optionally may be a flange or other relatively larger sized (e.g., diameter) feature to engage iliac bone to facilitate compression) and comprises at least one thread along at least a portion of its length (e.g., a screw). The inner member may be threaded along most or all of its length. In some embodiments the inner member is threaded in one or more of a distal end region (e.g., a distal third of the inner member), a proximal end region (e.g., a proximal third of the inner member), a central region, or any other combination that allows the outer member (e.g., sleeve) to be secured to the inner member (e.g., screw).

The outer member may include one or more inner threads that are shaped to interface with at least one of the outer thread(s) on the inner member. The mating threads may help secure the inner and outer members. The outer member may also include a head, flange or other larger dimensioned feature at a lateral end sized to engage iliac bone to facilitate the compression.

Alternatively, and as shown in FIG. 4, the implant may include (part of outer member or secured to it during the procedure) one or more of a flange, washer, or nut about the end of the outer member (any of which may be referred to as a stabilizer), wherein the stabilizer can be sized to engage the ilium, prevent axial movement, and facilitate and maintain compression across the joints and along the axis.

In some methods of implantation, the outer member may be advanced through one ilium (e.g. from the right in FIG. 4), across a first SI joint, toward and across the midline, and the inner member may be advanced from the other side through the other ilium, across the second SI joint, toward and across the midline and within the outer member. The threaded inner member may be rotated into the inner bore of the outer member, which may be threaded along at least part of its length. The inner threaded member may be rotated through the outer member until the desired compression is applied by the features at the implant ends that are sized and configured to engage the ilia, create and maintain the compression.

The outer sleeve in FIG. 4 (or other sleeve herein) may include any suitable feature of any of the outer sleeves described in WO/2020/168269 or WO/2021/108590, the entire disclosures of which are incorporated by reference herein for all purposes. For example and without limitation, outer sleeves herein may include outer threads along at least a portion of their lengths. Additionally, for example, outer sleeves herein may include fenestrations therein or therethrough that facilitate at least one of ingrowth, on-growth, or through-growth. Additionally, outer sleeves herein may be 3D printed (e.g. additive manufacturing), for example, which may increase the design options and provide better anchoring, stabilization, and/or facilitate better tissue growth.

As set forth herein, the implants described herein do not necessarily need to be adapted to apply compression. For example, the implant in FIG. 4 may be implanted across the pelvis in the position as shown, and the inner and outer members may be secured together via the threaded interface(s), but the implant may optionally not include any larger dimensioned feature(s) at the ends. These alternative implants may still be able to provide bilateral SI joint stabilization and/or fusion (e.g., due to the outer sleeve surface features, for example) while safely implanted across the corridor, but may not need compression to effectively treat the patient, depending on the needs of the treatment. Any of the suitably modifiable implants herein may thus be modified accordingly so as not to apply compression across the SI joints.

In the embodiment in FIG. 4, the inner member may be adapted to provide more strength to the implant than the outer member, and it may have a smaller diameter or outer profile than the outer member, similar to the implants in WO/2020/168269. Similarly, the outer sleeve may be configured to provide better anchoring and stabilization than the inner member, while together they can impart the desired characteristics of the overall implant.

In some embodiments of the implant in FIG. 4 (or any other implant herein), the inner member may be smooth in the central portion, which may strengthen the inner member within the central portion of the sacrum between the foramen.

In the embodiment in FIG. 4, even though the SI joints and the ilia are not shown, it is understood that the implant extends laterally through and across the SI joints, and through and out of the ilia, wherein the ends may be interfaced with an iliac surface.

In some embodiments, the composite implants described in WO/2020/168269 may be modified to have lengths and an outer profile that facilitate bilateral SI joint stabilization implantation from one side, being implanted across both SI joints and through the desired sacrum corridor.

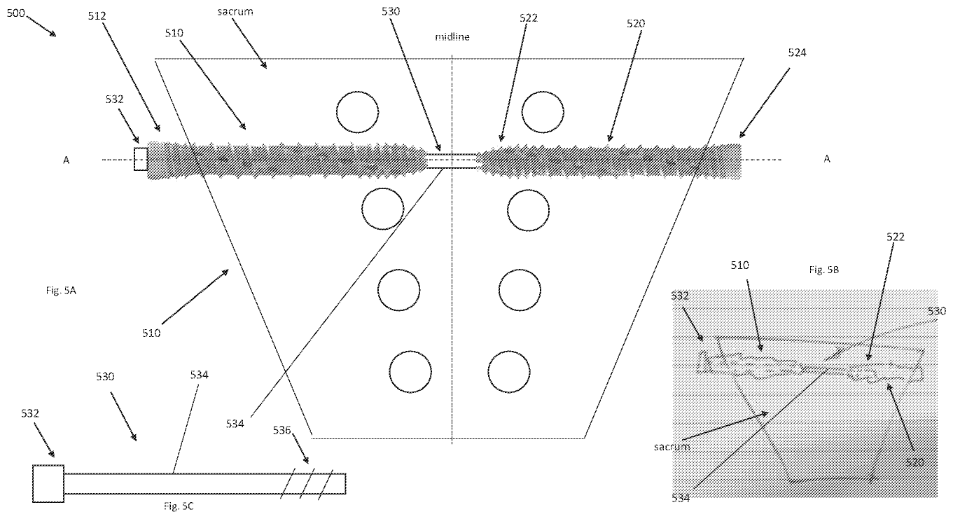

FIGS. 5A-5C illustrates an alternative approach to bilateral stabilization of the pelvis with compression along an implantation axis. In this approach, the implant 500 includes first and second implantable threaded members 510 and 520 that are each advanced from different sides through respective SI joints and into the sacrum along the corridor, in this example the S2 corridor. Implant 500 further includes an inner member 530. In general, one of the two threaded members (e.g., the threaded member 520) further includes internal threads in at least a distal region 522 that are configured to receive and mate with outer threads on a distal region of elongate inner member 530, which may be a threaded guide wire or pin. The first and second threaded member can be brought closer together, applying compression across the joints, by rotating elongate inner member 530 within the internally threaded member 520 such that the larger head 532 at the proximal end of elongate inner member 530 engages with the proximal end 512 of first implantable threaded member 510. In an exemplary implantation procedure, a pin (not shown) may be advanced into a first ilium (not shown for clarity), across a first SI joint (not shown for clarity), through the corridor, across the second SI joint, and into and through the second ilium along the desired trajectory. The first threaded member 520 may then be rotationally advanced into the ilium, across the SI joint, and into the sacrum (a broach may optionally be used to first create a bore). In this example, the first threaded member 520 is not advanced as far as the midline of the sacrum. The second threaded member 510 may then be rotationally advanced from the other side into the second ilium, across the second joint, and into the sacrum from the other direction (a broach may optionally be used to first create a bore). In this example, the second threaded member 510 is not advanced as far as the midline of the sacrum, as shown. By using a single pin, both implants can be delivered along a common axis (e.g., A-A). A guide pin may then be removed. Internal member 530, which includes an elongate shaft 534 and threads in at least a distal region, is then advanced through a lumen or channel within threaded member 510 (from left to right in FIG. 5), out the distal end of threaded member 510, and into the distal region 522 of threaded implant 520 where the outer threads on the inner member 530 will interface with the inner threads within implant 520. Inner member 530 may be rotated and advanced distally relative to both implants 510 and 530 until the larger proximal head 532 of the inner member 530 contacts the proximal end 512 of the first implant 510. Continued rotation of inner member 530 causes compression across the joint. Threaded implants 510 and 520 and inner member can then be left implanted in place to stabilize the pelvis and/or for SI joint fusion.

FIG. 5C illustrates exemplary inner member 530, including proximal head or enlarged region 532, elongate body 534, and threaded region 536 in at least a distal region of the elongate body 534.

The first and second members 510 and 520 may be, or include any suitable feature of, the threaded implants in WO/2021/108590.

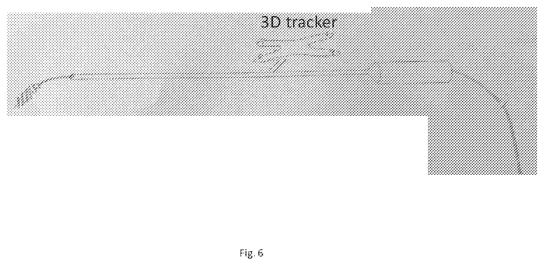

FIG. 6 illustrates an alternative general approach in which a threaded anchor that is coupled to a cable (or other similar elongate member) is delivered through a first ilium, across an SI joint, through the sacrum, across the contralateral SI joint, and into the contralateral ilium, where the anchor is fastened and implanted. Once the threaded anchor is anchored in position, the cable is tensioned on the entry side to apply the necessary compression, and a nut or other securing member is secured to the cable (not shown) to maintain the tension. In this example, the threaded anchor may be positioned using a 3D tracking and positioning system, examples of which are known for delivering medical devices. In this example, unlike FIG. 5, the implant includes a single threaded anchoring member.

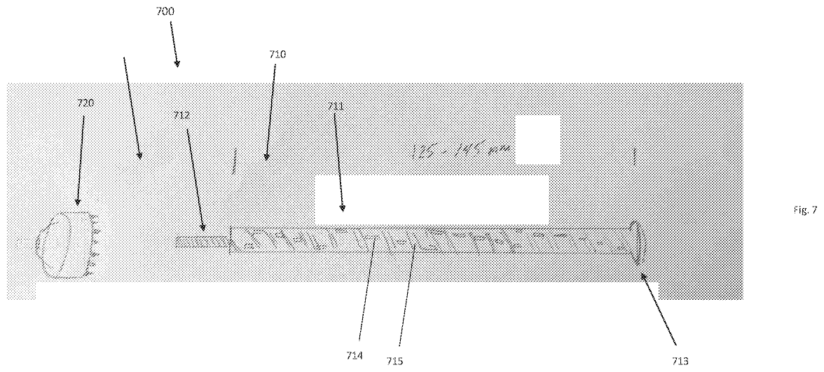

FIG. 7 illustrates an alternative system that is adapted for bi-lateral SI-joint placement and can be used to stabilize the pelvis, and which may be implanted in any of the locations described herein (e.g., along an axis that extends through an S1 corridor, an S2 corridor, or an S3 corridor). In general, implant or system 700 includes an elongate member 710 that is adapted to be advanced (in this example rotationally) through a first ilium, across an SI-joint, through a sacrum corridor, across the contralateral SI-joint, through the contralateral ilium until a distal end elongate member 710 is extending out of the contralateral ilium. Elongate member 710 includes an enlarged proximal end structure 713 (e.g., a flange) sized and configured to contact the first ilium and help facilitate compression. A securing member 720 (e.g., a nut) can be adapted to securely couple to the distal region 712 of elongate member 710, rotation of which relative to distal region 712 can be used to cause compression across both SI joints. Elongate member 710 may include threads 715 (only one section of which is labeled) along at least a portion of its length, including along all of or substantially all of its length. The main body 711 of the elongate member may include threads along any portion of its length as well as a plurality of fenestrations 714 (only one of which is labeled) distributed along a portion of its length. Body 711 may additionally include any features described in WO/2021/108590 A1, which is incorporated by reference herein. Body 711 may have variable features along its length. For example only, body 711 may be solid (lacking fenestrations) in a central region, with fenestrations in distal and proximal regions that are adjacent the central region. Alternatively, and for example only, body may be solid in a distal and/or proximal regions, and fenestrated in a central region.

In some embodiments, elongate member 710, including body 711, threaded distal extension 712, and proximal head 713 may be monolithic (e.g., formed from the same material), and may optionally be made with additive manufacturing techniques (e.g., 3D printing). In other embodiments, the elongate member may include multiple components that are secured together. For example, distal extension 712 may be an inner shank that extends through an outer body or sleeve 711. In some embodiments the implant 700, or at least elongate member 710, may have a length from 125 mm to 145 mm, which allows it to be implanted while the ends are disposed just outside of the ilia.

Alternatively, securing member 720 (e.g., a nut) may include a threaded male extension that extends within an internally threaded bore or channel in the distal end of body 711, wherein rotation of the securing member 720 creates compression across the joints.

While the term “implant” is used herein, it is understood that the implants may theoretically be removed after implantation, although this may be impractical to the extent in-growth, on-growth and/or through-growth has occurred.

The pelvic implants and systems in FIGS. 4-7 are examples of implants that comprise stabilization means for bilateral stabilization, and which optionally further comprise compression means for applying bilateral compression across first and second sacro-iliac joints.

Additionally, any of the implants herein may be made of material that adapts the implant to be resorbable over time. For example, when the implants provide stabilization during a healing process (e.g., from pelvic fracture), it may be acceptable or desirable that the implants resorb over time after the healing process. Without limitation, any of the implants herein, including any number of components, may be made from resorbable polymers.

1.-24. (canceled)

25. A method of one or both of pelvic stabilization or bilateral sacro-iliac joint stabilization, comprising:
laterally implanting an implant in a first ilium, across a first SI joint, through an S1, S2 or S3 sacrum corridor, across a contralateral second SI joint, and in a second ilium.

26. The method of claim 25, further comprising applying compression across the first and second SI joints with the implant.

27. The method of claim 25, wherein implanting the implant comprises advancing a first component of the implant from a first lateral side and advancing a second component of the implant from a second lateral side.

28. The method of claim 27, wherein implanting the implant further comprises engaging an outer external thread on the first component with an internal thread on the second component.

29. The method of claim 28, wherein when implanted, the second component extends across a midline of the sacrum.

30. The method of claim 29, wherein when implanted, the first component extends across the midline of the sacrum.

31. The method of claim 27, wherein the implanting step implants an entirety of the first component on a first side of a midline of the sacrum.

32. The method of claim 31, wherein the implanting step implants an entirety of the second component on a second side of a midline of the sacrum that is contralateral to the first side of the midline.

33. The method of claim 32, wherein the implanting step further comprises implanting a third component laterally from the first lateral side, through the first component, out of a distal end of the first component, and into engagement with the second component.

34. The method of claim 27, wherein implanting the implant further comprises implanting a third component from the first lateral side, through the first component, out a distal end of the first component, and into engagement with the second component.

35. The method of claim 34, further comprising engaging an outer thread on the third component with a second component inner thread.

36. The method of claim 34, wherein the first component and the second component have greater radially outermost dimensions from respective long axes than an outermost radial dimension of the third component.

37. The method of claim 27, wherein the first component has a greater radially outermost dimension than a radially outermost dimension of the second component.

38. A method of one or both of pelvic stabilization or bilateral sacro-iliac joint stabilization, comprising:
laterally implanting an implant in a first ilium, across a first SI joint, through an S1, S2 or S3 sacrum corridor, across a contralateral second SI joint, and in a second ilium,
wherein laterally implanting the implant comprises advancing a first component of the implant from a first lateral side and advancing a second component of the implant from a second lateral side.

39. The method of claim 38, further comprising applying compression across the first and second SI joints with the implant.

40. A pelvic implant, comprising:
stabilization means for bilateral stabilization that extends across first and second of a patient's sacro-iliac joints and through one of an S1 corridor, an S2 corridor, or an S3 corridor.

41. The pelvic implant of claim 40, wherein the stabilization means has a length from 125 mm to 145 mm.

42. The pelvic implant of claim 40, wherein the stabilization means comprises a first component and a second component, the first component having a distal end that is sized and configured to be advanced into a distal end of the second component and coupled thereto.

43. The pelvic implant of claim 42, wherein the first component includes an outer thread, and the second component comprises an internal thread.

44. The pelvic implant of claim 40 wherein the stabilization means comprises a first component, a second component, and a third component, wherein the third component is sized and configured to extend through the first and second components, and wherein a distal region of the third component is sized and configured to be coupled to an inner region of the second component.

45. The pelvic implant of 40, wherein the stabilization means further comprises compression means for applying compression between the first and second sacro-iliac joints.

46. A pelvic implant, comprising:
compression means for applying bilateral compression across first and second sacro-iliac joints and through one of an S1 corridor, an S2 corridor, or an S3 corridor that is between the sacro-iliac joints.
